(19) United States
(12) Patent Application Publication
(10) Pub. No.: US 2021/0174464 A1
(43) Pub. Date: Jun. 10, 2021

(54) Method of providing healthcare professionals to underserved communities

(71) Applicant: Olanshile Awanot Shitta-Bey, Chicago, IL (US)

(72) Inventor: Olanshile Awanot Shitta-Bey, Chicago, IL (US)

(21) Appl. No.: 15/932,880

(22) Filed: May 14, 2018

(51) Int. Cl. G06Q 50/20; G06Q 50/22; G06Q 20/08; G06Q 10/10; G16H 40/20; G06Q 30/02; G06Q 40/08; G09B 19/00

(52) U.S. Cl. CPC G06Q 50/2057; G06Q 50/22; G06Q 20/085; G09B 19/00; G16H 40/20; G06Q 30/0279; G06Q 40/08; G06Q 10/1053

(57) ABSTRACT

A method of placing a medical professional in an underserved community requiring medical professionals to provide medical care in the underserved community, by performing the steps of: collecting an application fee from a first plurality of applicants, the applicants being individuals qualified or graduate medical studies by having completed prerequisite medical training; providing profiles of applicants in the plurality to a second plurality of management professionals in communities that are underserved by medical professionals; receiving invitations from management professionals in the second plurality made to applicants in the first plurality, the invitations being invitations to train in an underserved community and make a commitment to provide medical services to the underserved community for a predetermined time period; and delivering the received invitations to intended applicants in the first plurality.

### FIELD OF THE INVENTION

The present invention relates broadly, and more specifically to finding opportunities for healthcare professionals to continue training and find work in the healthcare field.

### BACKGROUND OF THE INVENTION

Medical schools across the world produce a large number of graduates, some of whom have difficulty finding opportunities for residencies, internships, or other practical ways for gaining experience in the field in which so much of their efforts, time, and money were spent. Frustration is felt when, after the formal education ends, a recent graduate is unable to advance his or her career, and despite having valuable skills that could benefit others greatly if the graduate is given a chance, no opportunities are presented, and the recent graduate is forced to take employment unrelated to his or her training.

From another perspective, there are communities across the world that are underserved by medical professionals. While investments in facilities and equipment may have been adequately provided to these communities, skilled personnel remain a critical, yet persisting problem, for some. These communities do not have an easy way to make contact with individuals possessing the requisite skills, such as the recent medical graduate who cannot find a residency program described above.

### SUMMARY OF THE INVENTION

The present invention provides a solution to the problems described above by providing a matching service between underserved communities in need of medical professionals and medical professionals in need of an employment opportunity that provides continuing training and experience in the fields of medicine and healthcare.

In one aspect, the present invention provides a method of placing a medical professional in an underserved community requiring medical professionals to provide medical care in the underserved community, by: collecting an application fee from a first plurality of applicants, the applicants being individuals qualified for graduate medical studies by having completed prerequisite medical training; providing profiles of applicants in the plurality to a second plurality of management professionals in communities that are underserved by medical professionals; receiving invitations from management professionals in the second plurality made to applicants in the first plurality, the invitations being invitations to train in an underserved community and make a commitment to provide medical services to the underserved community for a predetermined time period; and delivering the received invitations to intended applicants in the first plurality.

In an embodiment, the invitations to train comprise invitations to participate in medical training focused on providing medical services to the underserved community.

In an embodiment, the method further includes providing the second plurality of management professionals with a training curriculum to be focused on the first plurality of applicants.

In an embodiment, providing the training curriculum includes obtaining funding from a nongovernmental organization.

In an embodiment, providing profiles to the second plurality of management professionals includes collecting fees from management professionals in the second plurality of management professionals.

In an embodiment, collecting fees comprises collecting a portion of a patient co-pay amount previously collected by a management professional in the second plurality.

In an embodiment, collecting fees comprises collecting a portion of an insurance payment amount previously collected by a management professional in the second plurality.

In an embodiment, the application fee is an annual fee.

In an embodiment, the profiles include an applicant's educational qualifications.

In an embodiment, the profiles include an applicant's work history.

Many additional features and advantages will become apparent to one skilled in the art upon reading the following detailed description, when considered in conjunction with the accompanying drawings, in which:

### BRIEF DESCRIPTION OF THE DRAWINGS

FIG. 1 is an architecture diagram showing a client server architecture implementing the present invention.

FIG. 2 is a logical sequence of steps illustrating the method of the present invention.

### DETAILED DESCRIPTION OF THE INVENTION

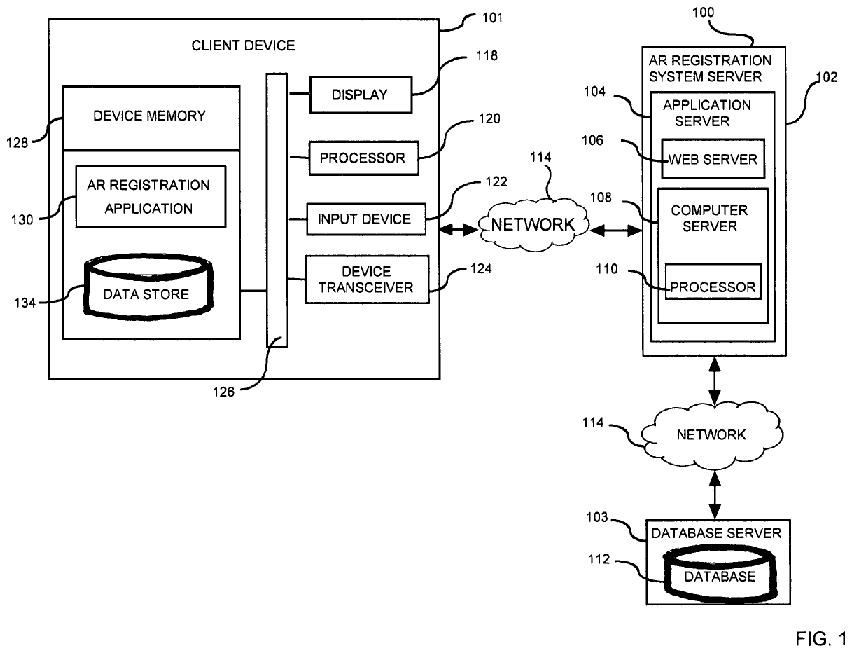

FIG. 1 illustrates the general architecture of a client-server application system 100 that operates in accordance with embodiments of the present invention. In a preferred embodiment, system 100 is implemented in multi-tier or n-tier architecture with one or more client devices 101 residing at the client tier, one or more servers 102 in the middle or server application tier and one or more database servers 103 residing in the database tier. In the above variant of three-tier architecture the client, the first tier, may have to only perform the user interface i.e., validate inputs; in which case the middle tier holds all the backend logic and does data processing while the data server, the third tier, performs data validation and controls the database access.

One or more client devices 101 are connected to system server 102 via a network 114. The system server 102 communicates with the client devices 101 over the network 114 to present a user interface or graphical user interface (GUI) for the service system 100 of the present invention. The user interface of the service system 100 of the present invention can be presented through a web browser or through a mobile application communicating with the system server 102 and is used for displaying, entering, publishing, and/or managing data required for the service. As used herein, the term “network” generally refers to any collection of distinct networks working together to appear as a single network to a user. The term also refers to the so-called world wide “network of networks” or Internet which is connected to each other using the Internet protocol (IP) and other similar protocols. As described herein, the exemplary public network 114 of FIG. 1 is for descriptive purposes only and it may be wired or wireless. Although the description may refer to terms commonly used in describing public networks such as the Internet, the description and concepts equally apply to other public and private computer networks, including systems having architectures dissimilar to that shown in FIG. 1. The inventive idea of the present invention is applicable for all existing cellular network topologies or respective communication standards, such as GSM, UMTS/HSPA, LTE and the like.

With respect to the present description, the system server 102 may include any service that relies on a database system that is accessible over a network, in which various elements of hardware and software of the database system may be shared by one or more users of the system 100. To this end, the users of the client device 101, from which a request or instruction is received over a network 114, may include any individual customer, a governmental or non-governmental organization, a group etc. The GUI or user interface provided by the system server 102 on the client devices 101 through a web browser or mobile app may be utilized by the users for utilizing service system 100.

The components appearing in system server 102 refer to an exemplary combination of those components that would need to be assembled to create the infrastructure to provide the tools and services contemplated by the present invention. As will be apparent to one skilled in the relevant art(s), all of components “inside” of system server 102 may be connected and may communicate via a wide or local area network (WAN or LAN).

The system server 102 includes an application server or executing unit 104. The application server or executing unit 104 comprises a web server 106 and a computer server 108 that serves as the application layer of the present invention. The Web server 106 is a system that sends out Web pages containing electronic data files in response to Hypertext Transfer Protocol (HTTP) requests from remote browsers (i.e. browsers installed in the client devices 101) or in response to similar requests made through a mobile app or mobile application of the present invention installed on a client device 101. The web server 106 can communicate with the mobile app of the present invention and/or with a web browser installed on a client device 101 to provide the user interface required for the service.

The computer server 108 may include a processor 110, a random-access memory (RAM) (not shown in figures) for temporary storage of information, and a read only memory (ROM) (not shown in figures) for permanent storage of information. Computer server 108 may be generally controlled and coordinated by operating system software. The operating system controls allocation of system resources and performs tasks such as processing, scheduling, memory management, networking, and I/O services, among (other) things. Thus, the operating system resides in system memory and, on being executed by CPU, coordinates the operation of the other elements of AR server 102.

Although the description of the computer server 108 may refer to terms commonly used in describing computer servers, the description and concepts equally apply to other processing systems, including systems having architectures dissimilar to that shown in FIG. 1.

The database tier is the source of data where at least one database server 103 generally interfaces multiple databases 112. Those databases are frequently updated by their users and administrators most often through a combination of private and public networks 114 including the Internet. It would be obvious to any person skilled in the art that, although described herein as the data being stored in a single database, different separate databases can also store the various data and files of multiple users.

A mobile application, or “app,” is a computer program that may be downloaded and installed in client device 101 using methods known in the art. Hereinafter, the mobile app 130 is referred to as app 130. App 130, custom built for the present invention, enables one or more persons to do various tasks related to live, in-person translation services using the system of the present invention. The activities related to the service of the present invention can also be performed using the user interface (or GUI) presented through a client device-based web browser. Hereinafter, the term “user interface” is used to refer to both app user interface and the web browser user interface of the present invention. Examples of client device 101 may include, but not limited to, mobile devices, tablets, hand-held or laptop devices, smart phones, personal digital assistants, desktop computers, wearable devices, augmented reality glasses, virtual reality headsets, or any similar device.

As illustrated in FIG. 1, the client device 101 may include various electronic components known in the art for this type of device. In this embodiment, the client device 101 may include a device display 118, a computer processor 120, a user input device 122 (e.g., touch screen, keyboard, microphone, and/or other form of input device known in the art), a device transceiver 124 for communication, a device memory 128, the AR app 130 operably installed in the computer memory 128, a local data store 134 also installed in the device memory 128, and a data bus 126 interconnecting the aforementioned components. For purposes of this application, the term “transceiver” is defined to include any form of transmitter and/or receiver known in the art, for cellular, WIFI, radio, and/or other form of wireless or wired communication known in the art. Obviously, these elements may vary, or may include alternatives known in the art, and such alternative embodiments should be considered within the scope of the claimed invention.

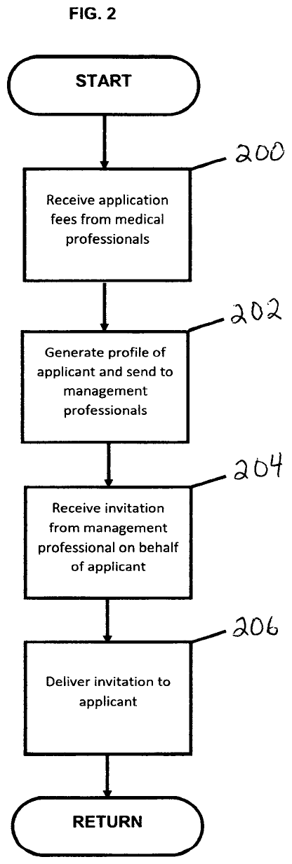

FIG. 2 shows a logical sequence of steps performed in accordance with the method of the present invention. The method shown in FIG. 2 places a medical professional in an underserved community requiring medical professionals to provide medical care in the underserved community, by collecting an application fee from a group of applicants at step 200. The applicants are individuals qualified for graduate medical studies by having completed prerequisite medical training. At step 202, profiles of applicants in the plurality to a second plurality of management professionals in communities that are underserved by medical professionals. Typically, the management professionals are hospital or clinic staff that make hiring decisions for a hospital or clinic. At step 204, invitations from management professionals are received on behalf of applicants having profiles in which the management professionals are interested in. The invitations are to train in an underserved community and make a commitment to provide medical services to the underserved community for a predetermined time period. At step 206, the received invitations are delivered to the intended applicants.

In an embodiment, the invitations to train comprise invitations to participate in medical training focused on providing medical services to the underserved community. In an embodiment, the method further includes providing the second plurality of management professionals with a training curriculum to be focused on the first plurality of applicants. In an embodiment, providing the training curriculum includes obtaining funding from a nongovernmental organization. In an embodiment, providing profiles to the second plurality of management professionals includes collecting fees from management professionals in the second plurality of management professionals.

In an embodiment, collecting fees comprises collecting a portion of a patient co-pay amount previously collected by a management professional in the second plurality. In an embodiment, collecting fees comprises collecting a portion of an insurance payment amount previously collected by a management professional in the second plurality. In an embodiment, the application fee is an annual fee. In an embodiment, the profiles include an applicant's educational qualifications. In an embodiment, the profiles include an applicant's work history.

As for interfaces, any screen that prompts a user to attach a document such as a resume or school transcripts, useful to management professionals in assessing an applicant's ability to serve the needs of an underserved community, can be used as an interface. As long as the applicant can attach documents to a message and send it to system server 102, the interface is sufficient. All other communication can be handled through simple message services or emails.

While a method for providing medical professionals has been described in the preferred embodiment and alternative embodiments, it is to be understood that numerous modifications can be made to the embodiments of the present invention without departing from the spirit thereof.

What is claimed is:

1. A method of placing a medical professional in an underserved community requiring medical professionals to provide medical care in the underserved community, by performing the steps of:
collecting an application fee from a first plurality of applicants, the applicants being individuals qualified for graduate medical studies by having completed prerequisite medical training;
providing profiles of applicants in the plurality to a second plurality of management professionals in communities that are underserved by medical professionals;
receiving invitations from management professionals in the second plurality made to applicants in the first plurality, the invitations being invitations to train in an underserved community and make a commitment to provide medical services to the underserved community for a predetermined time period; and
delivering the received invitations to intended applicants in the first plurality.

2. The method of claim 1, wherein the invitations to train comprise invitations to participate in medical training focused on providing medical services to the underserved community.

3. The method of claim 1, further comprising providing the second plurality of management professionals with a training curriculum to be focused on the first plurality of applicants.

4. The method of claim 3, wherein providing the training curriculum includes obtaining funding from a nongovernmental organization.

5. The method of claim 1, wherein providing profiles to the second plurality of management professionals includes collecting fees from management professionals in the second plurality of management professionals.

6. The method of claim 1, wherein collecting fees comprises collecting a portion of a patient co-pay amount previously collected by a management professional in the second plurality.

7. The method of claim 1, wherein collecting fees comprises collecting a portion of an insurance payment amount previously collected by a management professional in the second plurality.

8. The method of claim 1, wherein the application fee is an annual fee.

9. The method of claim 1, wherein the profiles include an applicant's educational qualifications.

10. The method of claim 1, wherein the profiles include an applicant's work history.
